(19) United States
(12) Patent Application Publication
(10) Pub. No.: US 2025/0009517 A1
(43) Pub. Date: Jan. 9, 2025

(54) TOTAL KNEE REPLACEMENT WITH COUPLABLE SPACERS

(71) Applicant: Erin E. Hofmann, Park City, UT (US)

(72) Inventor: Erin E. Hofmann, Park City, UT (US)

(21) Appl. No.: 18/219,620

(22) Filed: Jul. 7, 2023

Related U.S. Application Data
(60) Provisional application No. 63/339,723, filed on May 9, 2022.

(51) Int. Cl. A61F 2/30; A61F 2/00

(52) U.S. Cl. CPC A61F 2/30734; A61F 2/0095; A61F 2/30; A61F 2/30767; A61F 2002/30324; A61F 2002/30331; A61F 2002/30378; A61F 2002/30448; A61F 2002/30449; A61F 2002/30476; A61F 2002/3092; A61F 2002/3093

(57) ABSTRACT

The total knee replacement kit includes a femoral component, a tibial component, a patella component, and a set of couplable spacers selectively engageable with one another. The femoral component, the tibial component, and the patella in the kit are each selected based on a predetermined compatible size for implantation together as part of a total knee replacement system along with an implantation spacer that includes one of the set of couplable spacers in the kit having a desired thickness to position the tibial component from a tibia or multiple of the set of couplable spacers in the kit engaged with one another to achieve the desired thickness.

### BACKGROUND OF THE INVENTION

The present invention generally relates to a total knee replacement with couplable spacers. More specifically, the total knee replacement is a knee in a box that includes a femoral component, a tibial component, a patella, and multiple of the couplable spacers provided in a single box kit wherein multiple of the spacers may be removed from the box kit and coupled together to form a single relatively larger spacer without the need for traditional surgical trays having array of incrementally sized spacers.

Knee arthroplasties containing a femoral component, a tibial component, a patella, and a spacer are generally known in the art, wherein the femoral component is adapted for affixation onto the lower end of a patient femur and the patella is adapted to track along the femoral component as the patient bends their knee. The tibial component is adapted for affixation onto the upper end of the patient tibia and the spacer inserts between the tibial component and patient tibia to position the tibial component relative to the femoral component to best replicate natural articulation of the femoral component relative to the tibial component based on the anatomy of the patient knee joint. In a typical design, a single spacer formed from a biocompatible and high-density plastic material, such as polyethylene or the like, may be used, such as with the Zimmer Innex® Total Knee System manufactured and sold by Zimmer GmbH of Sulzerallee 8, 8484 Winterthur, Switzerland. As such, each component of the Zimmer Total Knee, including the spacers, may vary in size and thickness. Ultimately, the size selected by the surgeon depends on the anatomy of the patient. To accommodate patients that vary in size, surgeons performing knee arthroplasty procedures need access to differently sized spacers.

There exists, therefore, a need in the art for a sterilized total knee replacement that includes a preselected sized femoral component, tibial component, and patella combined with multiple couplable spacers in a single box kit, wherein the spacers may couple together to form a relatively larger spacer to meet surgical requirements more economically and efficiently for global delivery at a lower cost by reducing inventory.

### SUMMARY OF THE INVENTION

In one embodiment as disclosed herein, a set of couplable spacers for a total knee replacement system may include a first spacer having a first thickness and selectively implantable as a component within the total knee replacement system to position a tibial baseplate from a tibia by a first distance and a second spacer having a second thickness and selectively implantable as a component within the total knee replacement system to position the tibial baseplate from the tibia by a second distance. Here, the first spacer and the second spacer may be selectively engageable with one another prior to implantation to form a relatively thicker third spacer also selectively implantable as a component within the total knee replacement system to position the tibial baseplate from the tibia by a third distance relatively greater than the first distance and relatively greater than the second distance.

The first spacer and the second spacer may be of commensurate geometric shape, yet vary in the first thickness relative to the second thickness. This helps ensure compatibility with the total knee replacement system, namely from the standpoint that the implantable spacer varies only in its thickness as a result of stacking or selectively engaging multiple of the set of couplable spacers together. The first spacer and the second spacer may also include an outer periphery keyed for select snap-fit engagement with the other to ensure proper orientation. An adhesive may also be used to adhere the first spacer to the second spacer. In one embodiment, the adhesive may be a bone cement, fibrin adhesive, collagen adhesive, polyurethane, epoxy resin, cyanoacrylates, polyesters, polymethylmethacrylate, or zinc polycarboxylate. Additionally, each of the first spacer and the second spacer may include a porous coating at least partially disposed along an outer surface thereof.

Moreover, the first spacer may also selectively engage the second spacer in only one orientation determined by a keyed configuration or interface. Such a keyed interface helps ensure that the spacers are not coupled to one another in the wrong orientation. Specifically in this respect, each of the first spacer and the second spacer may include at least one recess and at least one protrusion. Here, the at least one protrusion of one of the first spacer or the second spacer is of a size and shape for select at least partial insertion into the recess of the other of the first spacer or the second spacer. As such, the first and second spacers may engage by way of snap-fit engagement between the recess and the protrusion. In one embodiment, each of the recesses may be formed from a bottom surface of the first spacer and the second spacer and each of the protrusions may extend up out from a top surface of the first spacer and the second spacer. Additionally, each recess of the first spacer and the second spacer may also include an inwardly projecting flange forming an open aperture relatively smaller than a top portion of the protrusion. As such, the top portion of the protrusion in the form of a bulbous head having a circumferential chamfered shoulder may be selectively positionable within an enclosure formed above the inwardly projecting flange of the recess, e.g., in snap-fit engagement therewith.

To facilitate keyed and properly oriented engagement, each of the recesses and the protrusions may be asymmetrically located on the first spacer and the second spacer. For example, in one embodiment, each of the spacers may include at least three recesses on a right side thereof and two recesses on a left side thereof. Similarly, each of the spacers may include a reciprocal set of three protrusions on the right side thereof and two protrusions on the left side thereof. Of course, each of the recesses and protrusions should be aligned for engagement with one another to facilitate keyed one-way engagement of one spacer with another. This, again, ensure proper key-fit orientation. Alternatively, the at least one recess may include multiple recesses with at least one of the multiple recesses having a circular cross-section and another of the multiple recesses having a rectangular cross-section. Similarly, and reciprocally, the at least one protrusion may include multiple protrusions with at least one of the multiple protrusions having a circular cross-section and another of the multiple protrusions having a rectangular cross-section.

In another embodiment as disclosed herein, a total knee replacement kit may include a femoral component, a tibial component, a patella component, and a set of couplable spacers selectively engageable with one another, wherein the femoral component, the tibial component, and the patella in the kit are each selected based on a predetermined compatible size for implantation together as part of a total knee replacement system along with an implantation spacer that may be one of the set of couplable spacers in the kit having a desired thickness to position the tibial component from a tibia or multiple of the set of couplable spacers in the kit engaged with one another to achieve the desired thickness.

In one embodiment, the set of couplable spacers may include includes at least two spacers having a thickness of 2 millimeters and 4 millimeters. In this embodiment, the implantation spacer in the kit would have thicknesses that include 2 millimeters, 4 millimeters, and 6 millimeters. As such, the kit has compatibility with three spacer thicknesses despite including only two spacer components therein. Alternatively, the set couplable spacers in the kit may include at least three spacers. Here, the first spacer may have a thickness of 1 millimeter, the second spacer may have a thickness of 2 millimeters, and the third spacer may have a thickness of 4 millimeters. As such, in this embodiment, the implantation spacer in the kit would have a thickness that includes 1 millimeter 2 millimeters, 3 millimeters, 4 millimeters, 5 millimeters, 6 millimeters, and 7 millimeters. To this end, the kit has compatibility with seven spacer thicknesses despite including only three spacer components therein. Of course, each of the set of couplable spacers may have the same or different thicknesses depending on the kit.

Additionally, the kit may further include a bearing component having a predetermined size and shape commensurate for implantation with the femoral component, the tibial component, and the patella component in the total knee replacement system. A sterile wrap may surround the total knee replacement kit to ensure the various components remain sterile inside and a lock may prevent each of the couplable spacers from sliding relative to one another when selectively engaged. Moreover, each of the set of couplable spacers may include a central aperture having a size and shape for select pass-through reception of an intramedullary stem of the tibial component to help ensure aligned coupling therewith. Alternatively, or in addition to, each of the set of couplable spacers may include a keyed aperture having a size and shape for pass-through reception of at least a portion of a downwardly projecting rod of the tibial component to help ensure aligned coupling therewith in one orientation. The kit may further include an adhesive having a chemical composition for bonding couplable spacers to one another. The adhesive may be used alone or in combination with a mechanical locking mechanism.

In accordance with another embodiment, a total knee replacement may include a femoral component, a tibial component, a patella, and one or more spacers capable of coupling together. Each of the spacers may include a top side or surface having one or more protrusions formed therein that generally orient toward a bottom side of the tibial component. In one embodiment, the protrusions may extend perpendicular to the top side of respective spacer. The spacers may also include a bottom side or surface having one or more commensurately positioned recesses formed therein and generally oriented toward a patient tibia. The recesses may include a lip that mates or otherwise receives a protrusion therein for snap-fit engagement or coupling therewith. Moreover, the protrusions and the recesses may be positioned in a keyed arrangement such that only one orientation of the spacers allows the mating of the protrusions of one spacer to the recesses of another spacer. The keyed arrangement, e.g., may include three protrusions/recesses on the left side of one spacer and two protrusions/recesses on the right side of another spacer, wherein each of the protrusions and recesses align to permit engagement (e.g., by snap-fit engagement) of the spacers. In one embodiment, the keyed arrangement of the protrusions/recesses may be in the form of any non-equilateral triangle. In alternative embodiments, the arrangement may be symmetrical.

In another embodiment, the top side of the spacers may include a protrusion or lip that generally follows the outer perimeter shape of the spacer to permit snap-fit engagement therealong with a commensurately formed recess in the bottom side of another spacer. In this embodiment, the perimeter geometry may facilitate engagement of the spacers in only in one orientation. As such, the spacers may couple together with or without the protrusion/recess combination, while maintaining a desired coupled orientation relative to one another. Alternatively, or in addition to, the spacers may couple together via one or more adhesives that may include bone cement, fibrin adhesive, collagen adhesive, polyurethane, epoxy resin, cyanoacrylates, polyesters, polymethylmethacrylate, or zinc polycarboxylate. Alternatively, or in addition to, the spacers may also couple together via a mechanical mechanism such as screws, staples, clips, or tape. Of course, any combination or adhesives, screws, protrusions/recesses, staples, clips, and/or tape may be used to couple one or more spacers as disclosed herein.

Another embodiment as disclosed herein may include a kit for use in performing a knee arthroplasty procedure. The kit may include a femoral component, a tibial component, and a patella of a predetermined or preselected size, all of which are combined in a container with multiple of the couplable spacers. While each of the femoral component, the tibial component, and the patella in the container or box may be utilized in surgery, the surgeon may select one or more of the couplable spacers for use in the surgery to attain the desired position of the tibial component relative to the femoral component. In this respect, in one embodiment, the box may include two of the couplable spacers, the first having a 2 millimeter (“mm”) thickness and the second having a 4 mm thickness. Here, the surgeon has the option of implanting one spacer with the 2 mm or 4 mm thickness, or may choose to couple the spacers together to form a single relatively larger spacer having a 6 mm thickness. As such, the two couplable spacers provides the surgeon three thickness options, namely 2 mm, 4 mm, and 6 mm. In another embodiment, the box may include two of the couplable spacers, the first having a 1 millimeter (“mm”) thickness and the second having a 2 mm thickness. Here, the surgeon has the option of implanting one spacer with the 1 mm or 2 mm thickness, or may choose to couple the spacers together to form a single relatively larger spacer having a 3 mm thickness. As such, the two couplable spacers provides the surgeon three thickness options, namely 1 mm, 2 mm, and 3 mm. In another embodiment, the box may include three of the couplable spacers, wherein one spacer may have a thickness of 1 mm, the second spacer may have a thickness of 2 mm, and the third spacer may have at thickness of 4 mm. Here again, the spacers may be used individually or combined together to attain the desired thickness. In this embodiment, the surgeon effectively has seven size options (i.e., 1 mm, 2, mm, 3 mm, 4 mm, 5 mm, 6 mm, and 7 mm) with only three physical spacers included in the box.

Other features and advantages of the present invention will become apparent from the following more detailed description, when taken in conjunction with the accompanying drawings, which illustrate, by way of example, the principles of the invention.

### BRIEF DESCRIPTION OF THE DRAWINGS

The accompanying drawings illustrate the invention. In such drawings:

FIG. 1 is an exploded perspective view of a total knee replacement kit including a femoral component, a tibial component, a patella component, one embodiment of a spacer as disclosed herein, all storable within a delivery box;

FIG. 2 is an anterior view of a patient knee joint;

FIG. 3 is an exploded perspective view of one embodiment of a total knee replacement system in accordance with the embodiments disclosed herein, including the femoral component, the patella component, a bearing component between the femoral component and the tibial component, and one spacer;

FIG. 4 is an exploded perspective view of another embodiment of the total knee replacement system in accordance with the embodiments disclosed herein, including the femoral component, the patella component, the tibial component, and a pair of spacers that couple together to form a single spacer having a thickness relatively greater than the one spacer of FIG. 3;

FIG. 5 is a side view of a pair of spacers in exploded relation relative to one another, with one spacer having a protrusion that engages a recess in the other spacer for snap-fit engagement therewith;

FIG. 6 is a side view similar to FIG. 5, further illustrating an adhesive disposed between the pair of spacers to enhance engagement therewith;

FIG. 7 is a side view illustrating coupling of the pair of spacers into a single spacer having a relatively larger thickness than either of the pair of spacers alone;

FIG. 8 is an alternative exploded perspective view of the exemplary total knee replacement, including the femoral component, the patella component, and the tibial component, for use with a set of three spacers that couple together form a single spacer having a relatively larger thickness than any of the three spacers alone;

FIG. 9 is a top plan view of an oval-shaped spacer having a plurality of square protrusions arranged in a non-equilateral triangle configuration;

FIG. 10 is a top plan view of the oval-shaped spacer similar to FIG. 9, alternatively illustrating the plurality of square protrusions in a keyed configuration with three of the protrusions on a left side thereof and two of the protrusions on a right side thereof;

FIG. 11 is a top plan view of the oval-shaped spacer of FIGS. 9 and 10, alternatively illustrating a plurality of circular protrusions arranged in the non-equilateral triangle configuration;

FIG. 12 is a top plan view of a rectangular spacer having a plurality of the square protrusions arranged in the non-equilateral triangle configuration; and

FIG. 13 is a top plan view of the rectangular spacer of FIG. 12, alternatively illustrating the plurality of square protrusions arranged in the keyed configuration with three of the protrusions on the left side thereof and two of the protrusions on the right side thereof.

### DETAILED DESCRIPTION OF THE PREFERRED EMBODIMENTS

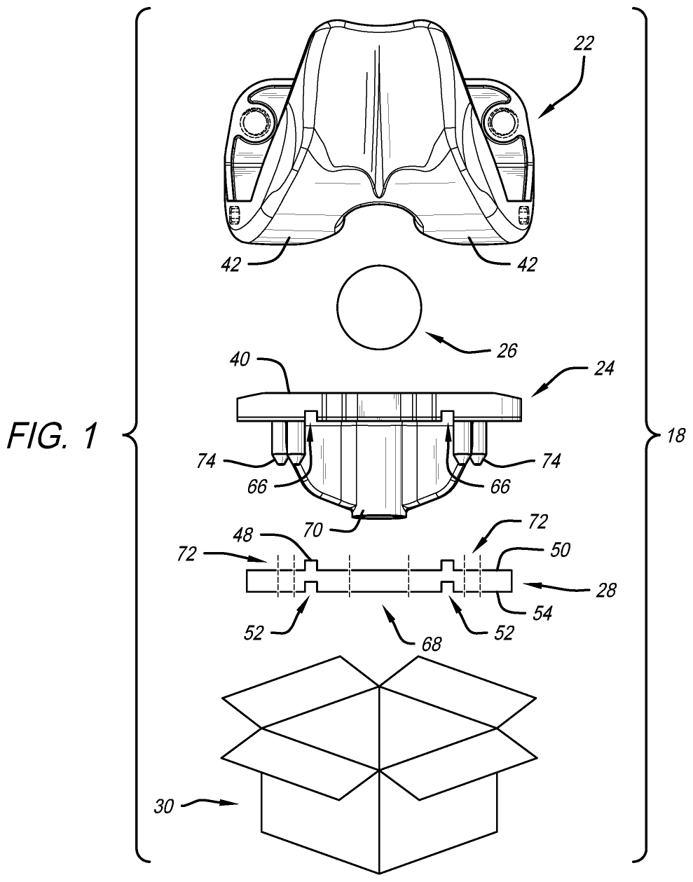

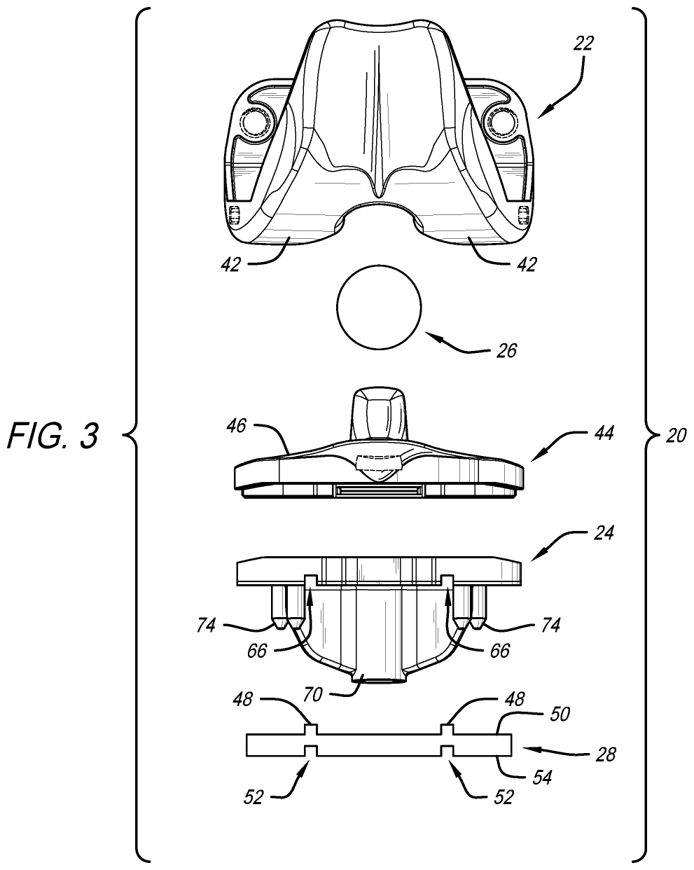

As shown in the exemplary drawings for purposes of illustration, a total knee replacement kit is illustrated in FIG. 1 with respect to reference numeral 18, while the corresponding total knee replacement system having one or more couplable spacers as disclosed herein is generally referred to in FIGS. 3, 4, and 8 with respect to reference numeral 20. In general, as illustrated in FIGS. 3, 4 and 8, the total knee replacement system 20 may include general components having a preselected or predetermined size, namely a femoral component 22 (e.g., manufactured from a metal material such as titanium or cobalt chrome, or a polymer material such as polyetheretherketone (“PEEK”)), a tibial component 24 (e.g., manufactured from polyethylene, ultra-high molecular weight polyethylene (“UHMWP”) or the like), a patella component 26, and/or one or more spacers 28, all of which may be housed and transported within a delivery box 30 as illustrated in FIG. 1. Each of the implant components 22, 24, 26, and the spacer 28 may be sterilized individually and then inserted within the separately sterilized box 30. Alternatively, the implant components 22, 24, 26, and the spacer 28 may first be placed into the box 30 and then all of the components 22, 24, 26, and the spacer 28 inside may be sterilized simultaneously. After sterilization, the box 30 may be wrapped to further maintain sterilization. As such, a single total knee replacement system 20 may be provided as the kit 18 within one box 30. During surgery, e.g., the surgeon may then use all or substantially all of the implant components 22, 24, 26, and the spacers 28, depending on the patient anatomy. The total knee replacement system 20 in the box 30 may thus reduce the expense of sterilizing, wrapping, and transporting incrementally sized components 22, 24, 26 in a tray, since many of the incrementally sized components 22, 24, 26 typically go unused during a conventional surgical procedure. In the event not all the spacers 28 are used, the unused spacer 28 may be discarded and, because all of the implant components 22, 24, 26 are otherwise implanted during the surgery, there is no need to re-sterilize and/or re-wrap leftover implant components, thereby further reducing surgical costs. As such, the “knee in a box” concept disclosed herein is tremendously cost effective over current practices for delivering implant components to an operating room.

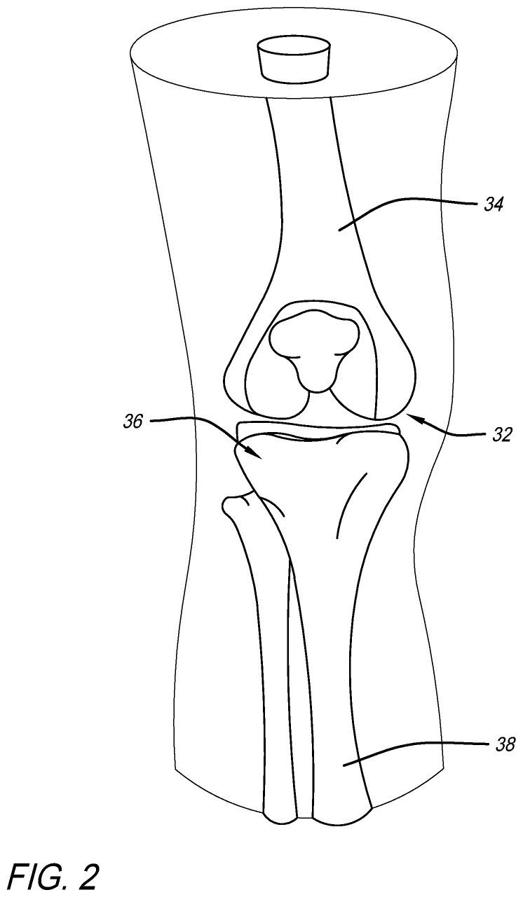

The femoral component 22 may generally affix or attach to a lower or distal end 32 of a femur 34, the tibial component 24 may generally affix or attach to an upper or proximal end 36 of a tibia 38, as illustrated in FIG. 2, and the patella component 26 may generally track along the femoral component 22 as the patient bends their knee. The femoral component 22 and/or the tibial component 24 may attach to the respective femur 34 and/or the tibia 38 via a suitable adhesive, such as bone cement like polymethylmethacrylate (“PMMA”) or the like. Alternatively, a portion of the femoral component 22 and/or the tibial component 24 may include a porous coating (not shown) in areas of contact with the respective femur 34 and/or the tibia 38, which promotes boney ingrowth and attachment thereto. Upon implantation, the femoral component 22 may be affixed to the femur 34 and the tibial component 24 may be affixed to the tibia 38 whereby the tibial component 24 will need to be positioned relative to the femoral component 22 to best replicate natural knee articulation in the total knee replacement system 20.

In this respect, the tibial component 34 may include an upper articular surface 40 having a size and shape generally commensurate with that of a pair of downwardly positioned condyles 42 of the femoral component 22, wherein each of the condyles 42 have a geometry that promotes or simulates natural knee movement or articulation. Of course, the femoral component 22 and the corresponding condyles 42 may be symmetric or asymmetric. Moreover, to attain such natural articular motion, the tibial component 24 should be positioned an appropriate distance from the femoral component 22. Here, the spacer 28 may be inserted between the tibial component 24 and tibia 38 to effectively raise the tibial component 24 upwardly toward the femoral component 22 for appropriate positioning thereunder. As such, the spacer 28 can be particularly useful in attaining the desired offset between the tibial component 24 and the femoral component 22 to maximize natural articular motion relative thereto.

FIG. 3 is an exploded perspective view illustrating an embodiment wherein a bearing component 44 (e.g., made of high-density plastic such as polyethylene or the like) is generally positioned between the femoral component 22 and the tibial component 24. The bearing component 44 may include an upper articular surface 46 having a size and shape generally commensurate with that of the pair of downwardly positioned condyles 42 of the femoral component 22. In this embodiment, the spacer 28 may be inserted between the tibial component 24 and the tibia 38 to effectively raise the tibial component 24 and the bearing component 44 upwardly toward the femoral component 22 for appropriate positioning thereunder. The bearing component 44 may be housed and transported within the box 30 (FIG. 1) along with the other implant components 22, 24, 26, and one or more of the spacers 28. As such, the bearing component 44 may be sterilized separately or simultaneously with the implant components 22, 24, 26, the spacer 28, and/or the box 30.

In conventional surgeries, numerous spacers may be delivered to the operating room whereby the surgeon may be able to select the desired spacer thickness depending on the anatomy of the patient. Although, in some circumstances, it may be desirable to substantially reduce the number of implant components in the operating room and/or otherwise at the point of surgery. In one embodiment, this could be accomplished by way of providing the femoral component 22, the tibial component 24, and/or the patella component 26 of preselected or predetermined size in a single kit or box 30, whereby the kit or box 30 includes all needed implant components 22, 24, 26 to perform a total knee arthroplasty. While the surgeon may be able to suitably approximate and select the size of each of the components 22, 24, 26 based on the anatomy of the patient before surgery or immediately before implantation, such a kit or box 30 containing the components 22, 24, 26 may still require inclusion of multiple of the spacers 28 so the surgeon can better perfect the location of the femoral component 22 relative to the tibial component 24. As such, in embodiments where a single set of commensurately sized components 22, 24, 26 are included in a single kit or box 30, reducing the number of spacers included therein will ensure the kit or box 30 remains relatively compact. In this respect, including multiple of the couplable spacers 40 as disclosed herein in the kit or box 30 ensures the container remains as compact as possible while, at the same time, maintaining the flexibility of the number of available spacer thicknesses to attain the desired positioning of the tibial component 24 relative to the femoral component 22 during surgery. Moreover, in another alternative embodiment, the tibial component 24 in the box 30 may be one size smaller than the femoral component 22 to match the radius of curvature to a ratio of about 1:1. In another embodiment, a size three femoral component 22 may be included and sterilized with a size three tibial component 24, a size two patella component 26, and a pair of the spacers 28 with the box 30.

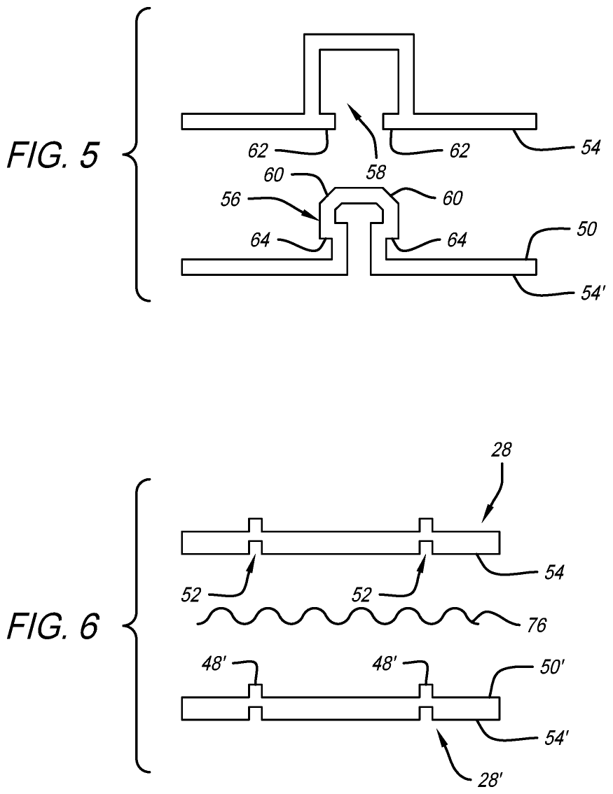

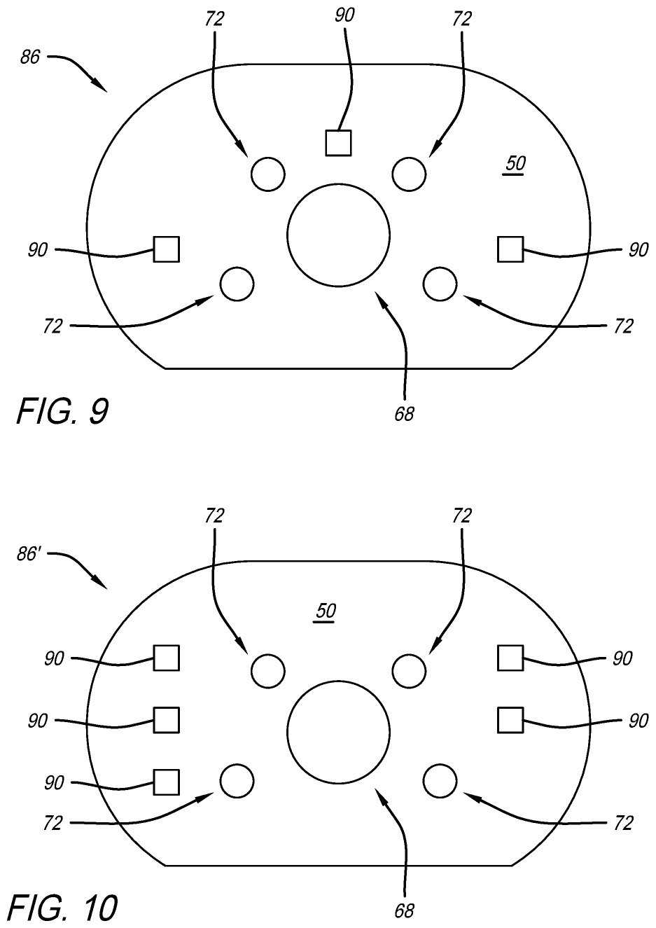

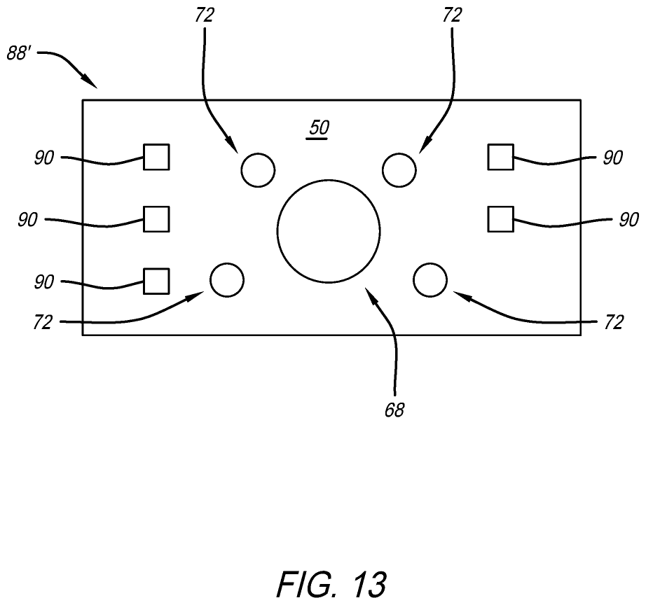

In one embodiment, FIGS. 1, 3, 4 and 8 illustrate that the spacer 28 may include at least one protrusion 48 upwardly projecting from a top surface 50 thereof and at least one indentation or recess 52 formed from a bottom surface 54 thereof, which may be of a size and shape to facilitate mating engagement with the protrusion 48 as discussed in more detail below. In general, the protrusions 48 and/or the recesses 52 may be formed of different geometric shapes such as oval (FIGS. 9-11), rectangular (FIGS. 12-13), square, triangular, hexagonal, octagonal, star-shaped, etc. Here, FIGS. 1, 3, 4, and 6-8 illustrate that the outer sidewalls of the protrusions 48 and that the inner cavities of the recesses 52 are generally smooth or straight. Although, in alternative embodiments, e.g., as illustrated in FIG. 5, the outer sidewalls of the protrusions 48 and/or the inner cavity of the recesses 52 may be mushroomed, ridged, grooved, corrugated, including a locking ledge, bumpy, rough, or uneven, such as to promote engagement therewith.

For example, FIG. 5 is an enlarged side view of one embodiment of a keyed protrusion 56 that may engage a pocketed recess 58 by snap-fit engagement. More specifically, FIG. 5 illustrates that the keyed protrusion 56 extends upwardly from the top surface 50 of one spacer and the pocketed recess 58 is formed from the bottom surface 54 of another spacer, in a similar manner as the protrusion 48 extends upwardly from the top surface 50 of the spacer 28 and the recess 52 is formed from the bottom surface 54 of the spacer 28 illustrated in FIG. 1. Although, here the keyed protrusion 56 includes a pair of chamfered upper sidewalls 60 that facilitate slide-through passage of the keyed protrusion 56 into the pocketed recess 58 by way of a pair of inwardly extending lips 62. The inwardly extending lips 62 may deflect to accommodate passthrough reception of the chamfered upper sidewalls 60. Once therein, a pair of outwardly extending shoulders 64 formed from the keyed protrusion 56 may seat within an interior of the pocketed recess 58 on top of the inwardly extending lips 62. This effectively mechanically holds or retains the keyed protrusion 56 within the pocketed recess 58. In one embodiment, one or more of the chamfered upper sidewalls 60, the inwardly extending lip 62, and/or the outwardly extending shoulders 64 may be circumferentially formed about one or more of the respective keyed protrusion 56 and/or the pocketed recess 58. This embodiment may provide enhanced engagement between the respective keyed protrusion 56 and the pocketed recess 58 by increasing the relative surface area engagement therebetween. As such, multiple of the spacers 28 may couple together via the keyed protrusion 56 of one spacer that matingly engages pocketed recess 58 of another spacer.

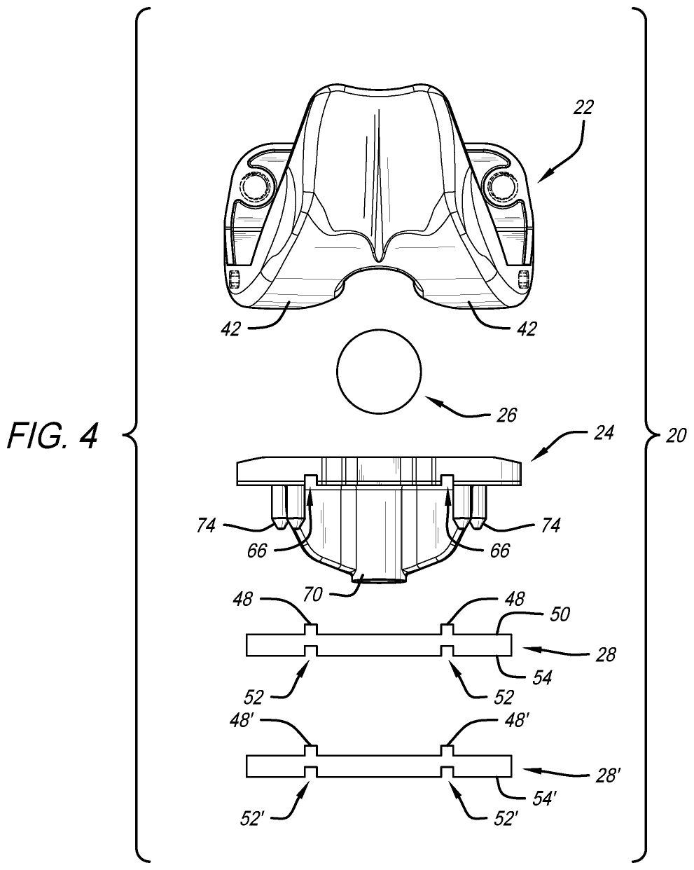

FIG. 4 is an exploded perspective view illustrating engagement of multiple of the spacers 28, 28′ in accordance with the embodiments disclosed herein. More specifically, FIG. 4 illustrates that the total knee replacement system 20 may include the aforementioned femoral component 22, the tibial component 24, and the patella component 26, but a pair of the spacers 28, 28′ are positioned underneath the tibial component 24 (and above the tibia 38 (FIG. 2)) to position the tibial component 24 at a relatively higher position than that illustrated in FIG. 1. Each of the spacers 28, 28′ include a respective pair of the protrusions 48, 48′ and a respective pair of the recesses 52, 52′. As discussed above, each of the protrusions 48′ upwardly extending from the spacer 28′ are of a size and shape for select reception and engagement with each of the recesses 52 formed from the spacer 28. As such, each of the spacers 28, 28′ are able to engage one another to form a “single” spacer having a relatively larger thickness. The use of the combination of the protrusions 48 and the recesses 52 may help facilitate locking engagement of one of the spacers 28, 28′ relative to the other spacer 28, 28′ so as to prevent one of the spacers 28, 28′ slipping or sliding relative to the other spacer 28, 28′. The combination of the protrusions 48 and the recesses 52 may also help facilitate aligned slide-on engagement of each of the spacers 28, 28′ with each other. As such, e.g., in this embodiment, the spacer 28 and the spacer 28′ may be generally identical in size, shape, and thickness. Although, of course, the spacers 28, 28′ may vary in thickness. Friction between the outer wall of the protrusions 48′ and the inner wall of the recesses 52 may help retain the spacers in locking engagement relative to one another as well.

Moreover, each of the upwardly extending protrusions 48 in the spacer 28 may engage (e.g., slide in reception) channels 66 in the tibial component 24 for engagement therewith. The spacer 28 may also have a central aperture 68 (best illustrated in FIGS. 1 and 9-13) that allows pass through reception of an intramedullary stem 70 of the tibial component 24. This allows the intramedullary stem 70 of tibial component 24 to extend through the central aperture 68 of the spacer 28 and generally insert or seat onto the tibia 38. The spacer 28 may also include a set of apertures 72 (FIGS. 1 and 9-13) that allow pass through reception of one or more downwardly projecting rods 74 (FIGS. 1, 3, 4, and 7) of the tibial component 24. The downwardly projecting rods 74 may extend through apertures 72 of the spacer 28 and generally insert or seat onto the tibia 38. Inserting the intramedullary stem 70 and/or downwardly projecting rods 74 into the tibia 38 anchors the tibial component 28 to the tibia 38 to increase stability of the total knee replacement system 20. Each of the spacer 28′, the recesses 52′, the bottom surface 54′, the intramedullary stem 70, and/or the downwardly projecting rods 74 may generally affix or attach to the tibia 38 via a suitable adhesive such as bone cement like polymethylmethacrylate (“PMMA”) or the like. Alternatively, a portion of the spacer 28′, the recesses 52′, the bottom surface 54′, the intramedullary stem 70, and/or the downwardly projecting rods 74 may include a porous coating (not shown) in areas of contact with the tibia 38, which promotes boney ingrowth and attachment thereto.

In alternative embodiments, the protrusions 48 of spacer 28 may downwardly project from the bottom surface 54, and the recesses 52 may be formed from the top surface 50. In this embodiment, the tibial component 24 may have downwardly projecting protrusions (not shown) that may engage (e.g., for slide-in reception) the recesses 52 in the top surface 50. Accordingly, the recesses 52′ of the spacer 28′ may engage with the downwardly projecting protrusions 48 of the spacer 28 to form the “single” spacer. In this embodiment, the downwardly projecting protrusions 48′ and/or the bottom surface 54′ of spacer 28′ may generally affix or attach to the tibia 38 via the aforementioned polymethylmethacrylate (“PMMA”) or porous coating (not shown).

Further to FIG. 4, FIG. 6 more specifically illustrates an embodiment wherein the first or top spacer 28 may couple or otherwise attach to the second or bottom spacer 28′ by way of an adhesive 76 disposed in between. The adhesive 76 may be applied to the bottom surface 54 of the top spacer 28, the top surface 50′ of the bottom spacer 28′, or the adhesive 76 may be applied to both the bottom surface 54 of the top spacer 28 and the top surface 50′ of the bottom spacer 28′. Pressing the bottom spacer 28′ into the top spacer 28 with the adhesive 76 disposed in between may help facilitate affixing the top spacer 28 to the bottom spacer 28′ by causing the adhesive 76 to extend into the recesses 52 of the top spacer 28 for enhanced engagement with the upstanding protrusions 48′ of the bottom spacer 28′. When dry, the adhesive 76 may lock the top spacer 28 to the bottom spacer 28′. The adhesive 76 may be a suitable bone cement such as polymethylmethacrylate (“PMMA”) or the like.

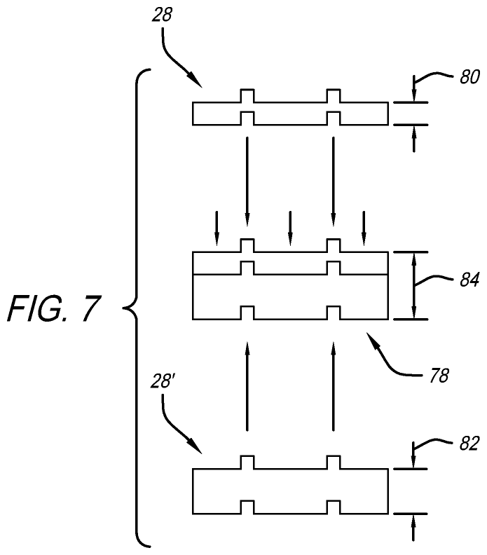

FIG. 7 illustrates affixing the top spacer 28 to the bottom spacer 28′ to form a single relatively larger spacer 78. More specifically, while not necessarily drawn to scale, FIG. 7 illustrates that the spacer 28 has a thickness 80 that is approximately one half the size of a thickness 82 of the spacer 28′. In one embodiment, the thickness 80 of spacer 28 may be 2 mm and the thickness 82 of spacer 28′ may be 4 mm. Here, when coupled, the individual spacers 28, 28′ may form the single relatively larger spacer 78 having a thickness 84 of approximately 6 mm, i.e., approximately 3 times that of the spacer 28, and approximately 50% larger than spacer 28′. Although, of course, in alternative embodiments, the thicknesses 80, 82 of each of the spacers 28, 28′ may be substantially the same. For example, in one embodiment, the thicknesses 80, 82 may be 2 mm and, when coupled together, the spacers 28, 28′ may form the single relatively larger spacer 78 having the thickness 84 of approximately 4 mm, i.e., approximately twice as thick as the individual spacers 28, 28′.

Although, of course, in alternative embodiments, the thicknesses 80, 82 may be different whereby the thickness 84 of the single relatively larger spacer 78 may be more than twice as thick as one of the spacers 28, 28′ and/or less than twice as thick as the other of the spacers 28, 28′. For example, the thickness 80 of the spacer 28 may be 1 mm while the thickness 82 of the spacer 28′ may be 3 mm. In this example, the thickness 84 of the relatively larger spacer 78 may be approximately 4 mm, whereby the single relatively larger spacer 78 is approximately 3 times thicker than the spacer 28 and only approximately 33% larger than the spacer 28′.

The ability to couple two or more of the spacers 28 together effectively decreases the number of spacers needed during surgery. For example, the box 30 may include three of the spacers 28 having respective thicknesses of 1 mm, 2 mm, and 4 mm. Here, by way of providing three spacers with varying thicknesses, the surgeon could effectively choose a desired thickness between 1 mm and 7 mm in 1 mm increments. In other words, three of the couplable spacers 28 provides the same options as conventional embodiments that require delivery of seven spacers to the operating room. As such, the couplable spacers 28 could effectively reduce the quantity of spacers delivered to the operating room by approximately 60%.

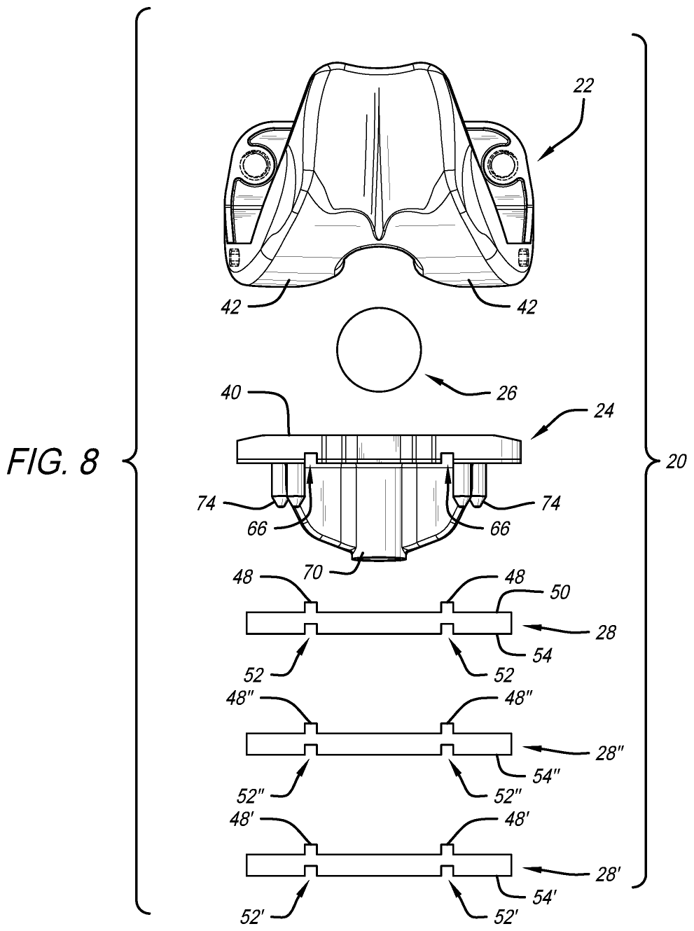

Moreover, FIG. 8 illustrates another embodiment of the total knee replacement system 20 incorporating the femoral component 22, the tibial component 24, the patella component 26, and three of the spacers 28, 28′, 28″. As shown, each of the spacers 28, 28′, 28″ are substantially the same construction, namely including at least a pair of the protrusions 48, 48′, 48″ and a pair of the recesses 52, 52′, 52″. The spacers 28, 28′, 28″ may also each have the central aperture 68 and the apertures 72 to allow pass through reception of the intramedullary stem 70 and downwardly projecting rods 74, respectively, as discussed, e.g., with respect to FIG. 1 above. Although, of course, each of the spacers 28, 28′, 28″ may be of different construction (e.g., more or less of the protrusions 48, 48′, 48″ and the recesses 52, 52′, 52″) so long as each of the spacers 28, 28′, 28″ are able to couple to one another and that the spacer 28 is able to couple to the tibial component 24 and the spacer 28′ is able to couple to the tibia 38. In accordance with the embodiments disclosed herein, the three spacers 28, 28′, 28″ may effectively couple together to form the single relatively thicker spacer 78 as discussed, e.g., with respect to FIG. 7 above. In an embodiment wherein each of the spacers 28, 28′, 28″ are of the same size and thickness (e.g., 2 mm), the single relatively larger spacer 78 will have the thickness 84 up to approximately 3 times larger (e.g., up to 6 mm) than each of the three spacers 28, 28′, 28″ alone.

The dimensions and number of the spacers 28 usable in accordance with the embodiments disclosed herein are for illustrative purposes only. Of course, the spacers 28 may be made of a variety of shapes and sizes, including different thicknesses; and the box or kit may include any number of the spacers 28. In one embodiment, the thickness of the spacers 28 may range from 1 to 10 mm and any number of the spacers 28 may couple together to attain the designed thickness for optimally locating the tibial component 24 relative to the femoral component 22. For example, instead of using three 2 mm thick spacers 28, 28′, 28″, two of the spacers 28, 28′ having 2 mm and 4 mm thicknesses may be combined to form the single relatively larger spacer 78 having a 6 mm thickness. In another example, two 3 mm thick spacers 28, 28′ may form the single relatively larger spacer 78 having the same 6 mm thickness. Alternatively, combining six of the spacers 28 having a 1 mm thickness may accomplish the same.

Since the femoral component 22, the tibial component 24, the patella component 26, and/or the bearing component 44 may come in different sizes, the combination of the spacers 28 used in connection with certain sizes of the femoral component 22, the tibial component 24, the patella component 26, and/or the bearing component 44 may also vary. In one embodiment, the number and size of the spacers 28 may be the same regardless of the size of the femoral component 22, the tibial component 24, the patella component 26, and/or bearing component 44. Alternatively, the number and size of the spacers 28 may vary in quantity and thickness depending on the size of the femoral component 22, the tibial component 24, the patella component 26, and or bearing component 44 to be included in the aforementioned box or kit. For example, fewer or smaller spacers 28 may be included with relatively smaller components 22, 24, 26, 44; and more or larger spacers 28 may be included with relatively larger components 22, 24, 26, 44.

Additionally, FIGS. 9-13 illustrate a variety of alternative embodiments of the spacer 28. For example, in one embodiment, FIGS. 9-11 illustrate a generally oval-shaped spacer 86, 86′, 86″. In this respect, the oval-shaped spacers 86, 86′, 86″ may vary in shape, e.g., being shaped more like a rectangle with rounded corners or more circular. Moreover, FIGS. 12 and 13 illustrate a relatively rectangular spacer 88, 88′. The oval-shaped spacers 86, 86′, 86″ and the rectangular spacers 88, 88′ are simply additional examples demonstrating that the spacers for use with the embodiments disclosed herein may vary in geometric shape and/or size, including depending on the desired application and fit.

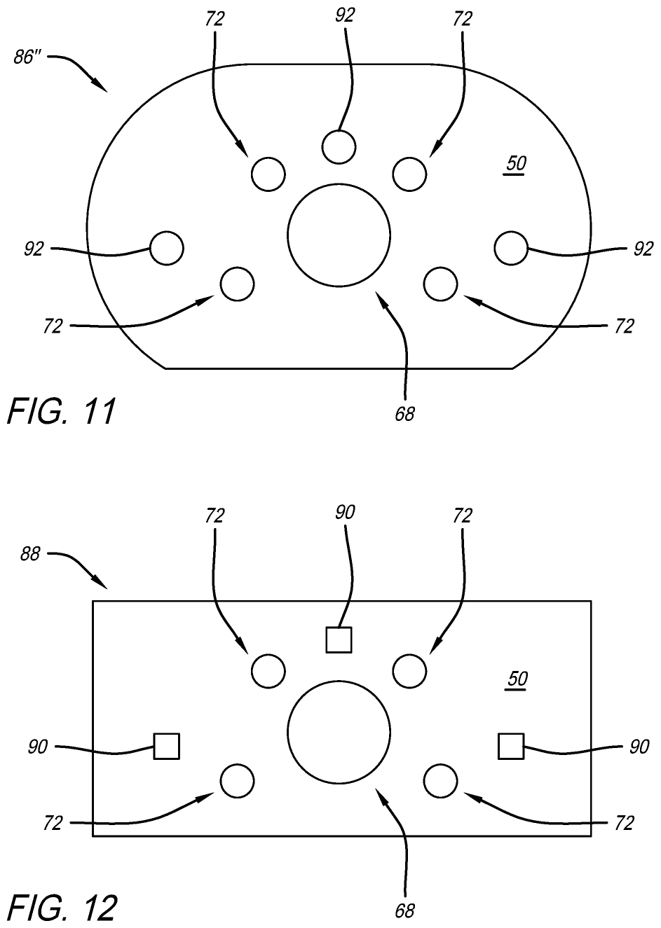

Moreover, FIGS. 9-13 also illustrate that the protrusions can vary in geometric shape and orientation/layout, namely FIGS. 9-10 and 12-13 illustrate a set of square protrusions 90 while FIG. 11 illustrates a set of circular or cylindrical protrusions 92, wherein each of the protrusions 90, 92 are formed from the top surface 50 of the respect to spacer 86, 86′, 86″, 88, 88′. Of course, in each embodiment, the oval-shaped spacers 86, 86′, 86″ and/or the rectangular spacers 88, 88′ may also include a set of complimentary square shaped recesses or rectangular shaped recesses extending into the bottom surface 54 thereof, and having a similar size and shape for select reception and engagement with the respective set of the protrusions 90, 92 as illustrated in FIGS. 9-13. Although, of course, the protrusions and/or the recesses may be any shape known in the art, so long as the protrusions and/or the recesses are compatible to inter-fit with one another.

Moreover, the protrusions 48, 90, 92 and/or any of the commensurately formed recesses 52 may be arranged in a keyed configuration such that the corresponding spacers that couple together inter-fit with one another in a single orientation. In other words, when the spacers are oriented correctly, the protrusions of one spacer will easily slidingly engage in mating relationship with the recesses of another spacer; but when the spacers are not oriented correctly, the spacers fail to engage one another. In one embodiment, e.g., illustrated in FIGS. 9, 11, and 12, each of the protrusions 90, 92 may be generally arranged in a symmetrical pattern that includes a non-equilateral triangle. Here, while the shape is symmetrical, the non-equilateral triangular orientation of each of the protrusions 90, 92 still requires one way engagement between adjacent spacers to be coupled together. As such, this facilitates one way or keyed engagement of the protrusions 90, 92 extending upwardly from the top surface 50 with the corresponding recesses formed from the bottom surface 54 to ensure that each of the coupled spacers are oriented correctly relative to one another. Of course, such keyed arrangement could also take on other configurations, including those illustrated in FIGS. 10 and 13. As shown, the embodiments illustrated in FIGS. 10 and 13 include a set of the protrusions 90 arranged in a manner locating three of the protrusions 90 on a left side of the respective spacers 86′, 88′ and locating two of the protrusions 90 on a right side of the respective spacers 86′, 88′. Such keyed arrangement may again ensure that the spacers couple together in a correct orientation relative to one another since there is effectively only one orientation where the protrusions 90 will engage the recesses 52. If, e.g., respective adjacent spacers, such as those illustrated in FIGS. 4 and 6-8 with respect to the spacers 28, 28′, 28″, are not in a common or desired orientation relative to one another, then the protrusions of one spacer will not align with the respective recesses in another spacer to permit slide-in engagement and spacer coupling. For example, when incorrectly oriented, any of the outwardly extending protrusions 48, 90, 92 will contact the bottom surface 54 as opposed to sliding into the recesses 52 for flush engagement therewith. Although, of course, the arrangements of any of the protrusions 48, 90, 92 may be symmetric, keyed, or in any desired configuration/orientation to facilitate coupling two or more spacers together, including in configurations that are not necessarily keyed.

Furthermore, FIGS. 9-13 also illustrate that the spacers 86, 86′, 86″, 88, 88′ may have the central aperture 68 and the apertures 72 to allow pass through reception of the intramedullary stem 70 and downwardly projecting rods 74, respectively. While FIGS. 9-13 illustrate that the central aperture 68 and/or the apertures 72 may be circular, the central aperture 68 and the apertures 72 may be any shape known in the art so long as to allow pass through reception of the intramedullary stem 70 and/or the downwardly projecting rods 74, respectively. Just as the protrusions 48, 90, 92 may be in a keyed configuration, the apertures 72 may also be arranged in a keyed configuration to ensure consistent one-way engagement. Here, while the apertures 72 of spacers 86, 86′, 86″, 88, 88′ are symmetric from left to right, they are asymmetric from top to bottom. This keyed arrangement ensures that the downwardly projecting rods 74 may only pass through the apertures 72 if the spacer 86, 86′, 86″, 88, 88′ is oriented correctly. For example, when incorrectly oriented, the downwardly projecting rods 74 may contact the top surface 50 of the spacer 86, 86′, 86″, 88, 88′ and prevent the downwardly projecting rods 74 from passing through the apertures 72. Consequently, this would also prevent the spacer 86, 86′, 86″, 88, 88′ from coupling to the tibial component 24. Although, of course, the arrangements of any of the central aperture 68 and the apertures 72 may be symmetric, keyed, or in any desired configuration/orientation to facilitate the spacer 28, 86, 86′, 86″, 88, 88′ coupling to the tibial component 24 and to facilitate coupling two or more of the spacers 28, 28′, 28″, 86, 86′, 86″, 88, 88′ together, including in configurations that are not necessarily keyed.

In alternative embodiments wherein the spacer does not include any of the protrusions 48, 90, 92 and/or any of the recesses 52, the size and shape of the respective spacer may facilitate desired engagement for purposes of coupling. For example, the geometry of the top surface 50 may coincide with the geometry of the bottom surface 54 to facilitate keyed engagement of one spacer to another. In another example, an outer periphery of the top surface 50 of one spacer may be configured for snap-fit engagement with an outer periphery of the bottom surface 54 of another spacer. This may couple adjacent spacers with or without the adhesive 76 disposed in between.

In general, the spacers may couple via at least one adhesive such as bone cement, fibrin adhesive, collagen adhesive, polyurethane, epoxy resin, cyanoacrylates, polyesters, polymethylmethacrylate, or zinc polycarboxylate and/or may couple to one another via at least one mechanical mechanism such as screws, staples, clips, tape, etc. In this respect, any combination or protrusions/recesses, adhesives, screws, staples, clips, and/or tape may be used to couple the two or more spacers together to form the single relatively larger spacer 74.

Moreover, and as briefly disclosed above, the box 30 may include each of the femoral component 22, the tibial component 24, the patella component 26, and multiple of the spacers 28. The components 22, 24, 26 and the spacers 28 may be contained in the box 30, or in a tray, bin, bag, case, or any comparable container, including one conducive for transportation and/or delivery to the operating room. The components 22, 24, 26 and/or the spacers 28 may also be arranged in the box 30 and sterilized simultaneously, as briefly mentioned above. In one example, the box 30 may include the femoral component 22, the tibial component 24, and the patella component 26 grouped together with two spacers 28 having respective thicknesses of 2 mm and 4 mm. This way, a surgeon performing a knee arthroplasty procedure with this box 30 may be able to position the tibial component 24 relative to the femoral component 22 by upwards of 6 mm, in increments of 2 mm. In another example, the box 30 may include the femoral component 22, the tibial component 24, and the patella component 26 grouped together with three spacers 28 having respective thicknesses of 1 mm, 2 mm, and 4 mm. This way, a surgeon performing a knee arthroplasty procedure with this kit may be able to position the tibial component 24 relative to the femoral component 22 by increments of 1 mm up to 7 mm.

Of course, the box 30 may include any of the components 22, 24, 26, 44, which may vary in size and combination. For example, in one embodiment, the tibial component 24 may have a 10, 12, 14, 16, or 18 mm thickness. In one example where the box 30 includes the tibial component 24 having a 12 mm thickness, but the surgical procedure requires a 14 mm thick tibial component 24, the surgeon may elect to couple the 2 mm spacer 28 to the tibial component 24 to increase the thickness thereof from 12 mm to 14 mm. The same box 30 may be used to perform surgeries where a 16 mm or 18 mm tibial component 24 is required by using the 4 mm spacer or the combined 2 mm and 4 mm spacers together to attain the desired thickness. Of course, the surgeon can use any combination of the spacers 28 to attain the desired offset. In this way, the box 30 may be generally usable with most surgeries regardless of the anatomy of the patient. This is because the size of the femur, tibial baseplate, and/or patella to be implanted can be fairly well approximated prior to surgery, while the distance the tibial baseplate needs to be positioned relative to the tibia cannot. As such, the surgeon can appropriately position the tibial baseplate using one box or kit with multiple spacers therein as opposed to needing entire trays to be delivered to the operating room to perform a total knee arthroplasty. Or course, in other applications, the size of the femoral component 22, the tibial component 24, and/or the patella component 26 included in any kit or box may vary in size along with the number and thickness of the spacers included therein.

Although several embodiments have been described in detail for purposes of illustration, various modifications may be made without departing from the scope and spirit of the invention. Accordingly, the invention is not to be limited, except as by the appended claims.

What is claimed is:

1. A set of couplable spacers for a total knee replacement system, comprising:
a first spacer having a first thickness and selectively implantable as a component within the total knee replacement system to position a tibial baseplate from a tibia by a first distance; and
a second spacer having a second thickness and selectively implantable as a component within the total knee replacement system to position the tibial baseplate from the tibia by a second distance, the first spacer and the second spacer being selectively engageable with one another prior to implantation to form a relatively thicker third spacer also selectively implantable as a component within the total knee replacement system to position the tibial baseplate from the tibia by a third distance relatively greater than the first distance and relatively greater than the second distance.

2. The set of couplable spacers of claim 1, wherein the first spacer and the second spacer comprise commensurate geometric shapes, varying only in the first thickness relative to the second thickness.

3. The set of couplable spacers of claim 1, wherein the first spacer selectively engages the second spacer in one orientation by a keyed configuration.

4. The set of couplable spacers of claim 1, wherein each of the first spacer and the second spacer include at least one recess and at least one protrusion, the at least one protrusion of one of the first spacer or the second spacer having a size and shape for select at least partial insertion into the recess of the other of the first spacer or the second spacer.

5. The set of couplable spacers of claim 4, wherein each of the recesses are formed from a bottom surface of the first spacer and the second spacer and each of the protrusions extend up out from a top surface of the first spacer and the second spacer.

6. The set of couplable spacers of claim 4, wherein each recess of the first spacer and the second spacer includes an inwardly projecting flange forming an open aperture relatively smaller than a top portion of the protrusion.

7. The set of couplable spacers of claim 6, wherein the top portion of the protrusion includes a bulbous head having a circumferential chamfered shoulder selectively positionable within an enclosure formed above the inwardly projecting flange of the recess.

8. The set of couplable spacers of claim 6, wherein each of the recesses and the protrusions are asymmetrically located on the first spacer and the second spacer.

9. The set of couplable spacers of claim 8, including at least three recesses on a right side and two recesses on a left side of each of the first spacer and the second spacer and at least three commensurate protrusions on the right side and two commensurate protrusions on the left side of each of the first spacer and the second spacer.

10. The set of couplable spacers of claim 4, wherein the at least one recess comprises multiple recesses with at least one of the multiple recesses comprising a circular cross-section and another of the multiple recesses comprising a rectangular cross-section, and the at least one protrusion comprises multiple protrusions with at least one of the multiple protrusions comprising a circular cross-section and another of the multiple protrusions comprising a rectangular cross-section.

11. The set of couplable spacers of claim 1, wherein each of the first spacer and the second spacer include an outer periphery keyed for select snap-fit engagement with the other.

12. The set of couplable spacers of claim 1, including an adhesive adhering the first spacer to the second spacer, the adhesive selected from the group consisting of bone cement, fibrin adhesive, collagen adhesive, polyurethane, epoxy resin, cyanoacrylates, polyesters, polymethylmethacrylate, and zinc polycarboxylate.

13. The set of couplable spacers of claim 1, wherein each of the first spacer and the second spacer include a porous coating at least partially disposed along an outer surface.

14. A total knee replacement kit, comprising:
a femoral component;
a tibial component;
a patella component; and
a set of couplable spacers selectively engageable with one another, wherein the femoral component, the tibial component, and the patella in the kit are each selected based on a predetermined compatible size for implantation together as part of a total knee replacement system along with an implantation spacer comprising one of the set of couplable spacers in the kit having a desired thickness to position the tibial component from a tibia or multiple of the set of couplable spacers in the kit engaged with one another to achieve the desired thickness.

15. The total knee replacement kit of claim 14, wherein the set of couplable spacers includes at least two spacers.

16. The total knee replacement kit of claim 15, wherein the first spacer comprises a thickness of 2 millimeters and the second spacer comprises a thickness of 4 millimeters, whereby the implantation spacer in the kit comprises a thickness of 2 millimeters, 4 millimeters, or 6 millimeters.

17. The total knee replacement kit of claim 14, wherein the set of couplable spacers includes at least three spacers.

18. The total knee replacement kit of claim 17, wherein the first spacer comprises a thickness of 1 millimeter, the second spacer comprises a thickness of 2 millimeters, and the third spacer comprises a thickness of 4 millimeters, whereby the implantation spacer in the kit comprises a thickness of 1 millimeter to 7 millimeters in 1 millimeter increments.

19. The total knee replacement kit of claim 14, wherein the kit further includes a bearing component having a predetermined size and shape commensurate for implantation with the femoral component, the tibial component, and the patella component in the total knee replacement system.

20. The total knee replacement kit of claim 14, including a sterile wrap surrounding the total knee replacement kit.

21. The total knee replacement kit of claim 14, wherein each of the set of couplable spacers comprise a different thickness.

22. The total knee replacement kit of claim 14, including a lock preventing each of the couplable spacers from sliding relative to one another when selectively engaged.

23. The total knee replacement kit of claim 14, wherein each of the set of couplable spacers include a central aperture having a size and shape for select pass-through reception of an intramedullary stem of the tibial component.

24. The total knee replacement kit of claim 14, wherein each of the set of couplable spacers include a keyed aperture having a size and shape for pass-through reception of at least a portion of a downwardly projecting rod of the tibial component in one orientation.

25. The total knee replacement kit of claim 14, wherein the kit further includes an adhesive having a chemical composition for bonding one of the set of couplable spacers to another of the set of couplable spacers.
